SEQUENCE LISTING (1 sequence)

SEQ ID NO: 1 - protein, 17 aa, Artificial Sequence
  Synthetic peptide
IDQQVLSRIKLEIKRCL

(19) United States
(12) Patent Application Publication
(10) Pub. No.: US 2020/0360466 A1
(43) Pub. Date: Nov. 19, 2020

(54) METHOD OF IMPROVING LOWER URINARY TRACT SYMPTOMS

(71) Applicant: Nymox Corporation, Nassau (BS)

(72) Inventor: Paul Averback, Nassau (BS)

(21) Appl. No.: 16/410,685

(22) Filed: May 13, 2019

(51) Int. Cl. A61K 38/10; A61P 13/08

(52) U.S. Cl. CPC A61K 38/10; A61P 13/08

(57) ABSTRACT

The embodiments include methods of improving the symptoms of mammals having LUTS, using compositions containing Fexapotide Triflutate and a pharmaceutically acceptable carrier. The method includes, but is not limited to, administering Fexapotide Triflutate intramuscularly, orally, intravenously, intrathecally, intratumorally, intranasally, topically, transdermally, etc., either alone or with a carrier to a mammal in need thereof.

### BACKGROUND

### 1. Field of the Embodiments

The embodiments include methods of improving lower urinary tract symptoms (LUTS), and more particularly improving obstructive voiding symptoms in patients with LUTS, using compositions containing compounds based on small peptides and a pharmaceutically acceptable carrier.

### 2. Description of Related Art

The European Association of Urology (EAU) and American Urological Association (AUA) guidelines define LUTS as storage (irritative) symptoms (daytime urinary frequency, urgency, and nocturia), voiding (obstructive) symptoms (straining, weak stream, intermittent stream, and incomplete emptying), or postmicturition symptoms (postmicturition dribbling) that affect the lower urinary tract (LUT). Oelke M, et al., European Association of Urology, Eur. Urol. 2013 July; 64(1):118-40.

Benign Prostatic Hyperplasia (BPH) is a histologic diagnosis that refers to the nonmalignant proliferation of smooth muscle and epithelial cells of the prostate. Lee C, et al., “Intrinsic and extrinsic factors controlling benign prostatic growth,” Prostate, 1997; 31:131-138; Auffenberg G B, et al., “Established medical therapy for benign prostatic hyperplasia,” Urol Clin North Am., 2009; 36:443-459. The exact etiology is unknown. The progression of BPH can lead to benign prostatic enlargement (BPE), which is determined by the size of the prostate (pathologic). Approximately 50% of men with histologic BPH develop BPE. BPE may eventually cause bladder outlet obstruction (BOO), which is also termed benign prostatic obstruction (BPO) if associated with BPE. BOO and BPO are determined with urodynamic measures. Some patients may present with BPE but not have significant LUTS, while other patients may present with LUTS and have a significant reduction in QoL but not have BPE. Park, H. J., et al., “Urinary Tract Symptoms (LUTS) Secondary to Benign Prostatic Hyperplasia (BPH)., World J. Mens Health, No. 31(3), 193-207 (2013).

Lower urinary tract symptoms (LUTS) generally are classified into 2 main types of symptoms: 1) “irritative” also referred to as “storage” symptoms; and 2) “obstructive” also referred to as “voiding” symptoms. The irritative/storage symptoms include urgency of need to urinate, higher frequency, and nocturia (need to urinate more frequently after going to sleep at night). The obstructive voiding symptoms include weak urinary stream, need to push or strain to evacuate the urine, sensations of incomplete emptying after urination, and stopping and starting several times during the course of voiding.

The United States and Europe have established guidelines to assist physicians in the treatment of LUTS, BPH, and LUTS/BPH. Oelke M, et al., European Association of Urology, Eur. Urol. 2013 July; 64(1):118-40. The guidelines discuss treatment options varying from watchful waiting (WW), for men presenting with symptoms but are not bothered enough to need medication or surgical intervention, to drug treatments, to surgical intervention. Drug treatment guidelines have included the use of alpha-blockers (alpha-adrenergic antagonists), 5-alpha-reductase inhibitors (5ARIs), antimuscarinics (anticholinergics), a PDE5 inhibitor (tadalafil), combination therapies, and vasopressin analogues. The use of combination therapies such as an alpha-blocker with a 5ARI or antimuscarinic also have been recommended.

Prostate surgery such as transurethral resection of the prostate is indicated in men with absolute indications or drug treatment-resistant LUTS. Indications for surgery include severe conditions such as urinary retention, gross hematuria, urinary tract infection, and bladder stones. Minimally invasive treatments suggested include transurethral microwave therapy and transurethral needle therapy. An alternative to catheterization for men unfit for surgery include prostate stents. Despite the various available treatment options, there remain unmet medical needs for effective and safe agents to treat these bothersome symptoms, some of which may be caused by prostate enlargement, which can lead to more serious problems such as chronic urinary tract infections, incontinence, acute and chronic urinary retention, and renal failure.

Some agents known to have the ability to destroy and hence either facilitate the removal of or inhibit the further growth of harmful or unwanted cells and tissue are disclosed in U.S. Pat. Nos. 6,924,266; 7,241,738; 7,317,077; 7,408,021; 7,745,572; 8,067,378; 8,293,703; 8,569,446; and 8,716,247; and U.S. Patent Application Publication Nos. 2017/0360885; 2017/0020957; 2016/0361380; and 2016/0215031, the disclosures of each of which are incorporated by reference herein in their entirety. One such agent is known as Fexapotide Triflutate.

There exists a need for treatments that can improve the symptoms of patients having LUTS without the risks and side effects of conventional drug therapies, or surgical intervention. There also exists a need for treatments that can improve the obstructive voiding symptoms of patients having LUTS without the risks and side effects of conventional drug therapies, or surgical intervention.

Throughout this description, including the foregoing description of related art, any and all publicly available documents described herein, including any and all U.S. patent published patent applications, are specifically incorporated by reference herein in their entirety. The foregoing description of related art is not intended in any way as an admission that any of the documents described therein, including pending U.S. patent applications, are prior art to the present disclosure. Moreover, the description herein of any disadvantages associated with the described products, methods, and/or apparatus, is not intended to limit the embodiments. Indeed, aspects of the embodiments may include certain features of the described products, methods, and/or apparatus without suffering from their described disadvantages.

### SUMMARY OF THE EMBODIMENTS

There remains a need in the art for new, less toxic, and less frequent (e.g., avoiding the need to take medications daily or weekly) treatments for improving the quality of life for patients with LUTS. There also remains a need in the art for such treatments that improve obstructive voiding symptoms in patients with LUTS. The embodiments satisfy these needs.

This disclosure is premised in part on the discovery that certain peptides, including a specific peptide described by the amino acid sequence Ile-Asp-Gln-Gln-Val-Leu-Ser-Arg-Ile-Lys-Leu-Glu-Ile-Lys-Arg-Cys-Leu, (Fexapotide Triflutate or “FT”) are capable of significantly improving lower urinary tract symptoms (LUTS), and more particularly improving obstructive voiding symptoms in patients with LUTS,

This disclosure also is premised in part on the discovery that the use of FT either alone or in combination with an additional active agent capable of treating and/or killing unwanted cellular proliferations in mammals, provides an unexpected improvement in patients having obstructive voiding symptoms associated with LUTS.

The compositions can be administered intramuscularly, orally, intravenously, intraperitoneally, intracerebrally (intraparenchymally), intracerebroventricularly, intratumorally, intralesionally, intradermally, intrathecally, intranasally, intraocularly, intraarterially, topically, transdermally, via an aerosol, infusion, bolus injection, implantation device, sustained release system etc. Alternatively, the FT peptide can be expressed in vivo by administering a gene that expresses the peptide, by administering a vaccine that induces such production or by introducing cells, bacteria or viruses that express the peptide in vivo, because of genetic modification or otherwise.

In another embodiment, administering a composition comprising FT, either alone or in combination with at least one additional active agent capable of treating and/or killing unwanted cellular proliferations in mammals improves the mean obstructive voiding symptoms (MOVS) measured by the International Prostate Symptom Score (IPSS), by more than 30%, when compared to administering a control composition that does not contain FT.

In another embodiment, administering a composition comprising FT, either alone or in combination with at least one additional active agent capable of treating and/or killing unwanted cellular proliferations in mammals improves the mean irritative storage symptoms (MISS) measured by the International Prostate Symptom Score (IPSS), by more than 20%, when compared to administering a control composition that does not contain FT.

Both the foregoing general description and the following detailed description are exemplary and explanatory and are intended to provide further explanation of the embodiments as claimed. Other objects, advantages, and features will be readily apparent to those skilled in the art from the following detailed description of the embodiments.

### DETAILED DESCRIPTION OF THE PREFERRED EMBODIMENTS

Before the embodiments are described, it is understood that this invention is not limited to the particular methodology, protocols, cell lines, vectors, and reagents described, as these may vary. It also is to be understood that the terminology used herein is for the purpose of describing particular embodiments only, and is not intended to limit the scope of the present embodiments which will be limited only by the appended claims.

Terms and phrases used herein are defined as set forth below unless otherwise specified. Throughout this description, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Amino acids and amino acid residues described herein may be referred to according to the accepted one or three-letter code provided in the table below.

TABLE 1

| Three-Letter Amino | One-Letter | |
|---|---|---|
| Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Fexapotide Triflutate (“FT”), as it is used herein, denotes a 17-mer peptide having the amino acid sequence: Ile-Asp-Gln-Gln-Val-Leu-Ser-Arg-Ile-Lys-Leu-Glu-Ile-Lys-Arg-Cys-Leu (SEQ ID NO. 1). FT is disclosed in U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031. The disclosures of these patents and published applications are incorporated by reference herein in their entirety. FT is represented by:

SEQ ID NO. 1:
IDQQVLSRIKLEIKRCL or Ile-Asp-Gln-Gln-Val-Leu-Ser-
Arg-Ile-Lys-Leu-Glu-Ile-Lys-Arg-Cys-Leu.

The term “fragment” refers to a protein or polypeptide that consists of a continuous subsequence of the amino acid sequence of a protein or peptide and includes naturally occurring fragments such as splice variants and fragments resulting from naturally occurring in vivo protease activity. Such a fragment may be truncated at the amino terminus, the carboxy terminus, and/or internally (such as by natural splicing). Such fragments may be prepared with or without an amino terminal methionine. The term “fragment” includes fragments, whether identical or different, from the same protein or peptide, with a contiguous amino acid sequence in common or not, joined together, either directly or through a linker. A person having ordinary skill in the art will be capable of selecting a suitable fragment for use in the embodiments without undue experimentation using the guidelines and procedures outlined herein.

The term “variant” refers to a protein or polypeptide in which one or more amino acid substitutions, deletions, and/or insertions are present as compared to the amino acid sequence of an protein or peptide and includes naturally occurring allelic variants or alternative splice variants of an protein or peptide. The term “variant” includes the replacement of one or more amino acids in a peptide sequence with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987.) Preferred variants include alanine substitutions at one or more of amino acid positions. Other preferred substitutions include conservative substitutions that have little or no effect on the overall net charge, polarity, or hydrophobicity of the protein. Conservative substitutions are set forth in Table 2 below.

TABLE 2
Conservative Amino Acid Substitutions

| | |
|---|---|
| Basic: | arginine |
| | lysine |
| | histidine |
| Acidic: | glutamic acid |
| | aspartic acid |
| Uncharged Polar: | glutamine |
| | asparagine |
| | serine |
| | threonine |
| | tyrosine |
| Non-Polar: | phenylalanine |
| | tryptophan |
| | cysteine |
| | glycine |
| | alanine |
| | valine |
| | praline |
| | methionine |
| | leucine |
| | isoleucine |

Table 3 sets out another scheme of amino acid substitution:

TABLE 3

| Original Residue | Substitutions |
|---|---|
| Ala | gly; ser |
| Arg | lys |
| Asn | gln; his |
| Asp | glu |
| Cys | ser |
| Gln | asn |
| Glu | asp |
| Gly | ala; pro |
| His | asn; gln |
| Ile | eu; val |
| Leu | ile; val |
| Lys | arg; gln; glu |
| Met | leu; tyr; ile |
| Phe | met; leu; tyr |
| Ser | thr |
| Thr | ser |
| Trp | tyr |
| Tyr | trp; phe |
| Val | ile; leu |

Other variants can consist of less conservative amino acid substitutions, such as selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to have a more significant effect on function are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine. Other variants include those designed to either generate a novel glycosylation and/or phosphorylation site(s), or those designed to delete an existing glycosylation and/or phosphorylation site(s). Variants include at least one amino acid substitution at a glycosylation site, a proteolytic cleavage site and/or a cysteine residue. Variants also include proteins and peptides with additional amino acid residues before or after the protein or peptide amino acid sequence on linker peptides. For example, a cysteine residue may be added at both the amino and carboxy terminals of FT in order to allow the cyclisation of the peptide by the formation of a di-sulphide bond. The term “variant” also encompasses polypeptides that have the amino acid sequence of FT with at least one and up to 25 or more additional amino acids flanking either the 3′ or 5′ end of the peptide.

The term “derivative” refers to a chemically modified protein or polypeptide that has been chemically modified either by natural processes, such as processing and other post-translational modifications, but also by chemical modification techniques, as for example, by addition of one or more polyethylene glycol molecules, sugars, phosphates, and/or other such molecules, where the molecule or molecules are not naturally attached to wild-type proteins or FT. Derivatives include salts. Such chemical modifications are well described in basic texts and in more detailed monographs, as well as in a voluminous research literature, and they are well known to those of skill in the art. It will be appreciated that the same type of modification may be present in the same or varying degree at several sites in a given protein or polypeptide. Also, a given protein or polypeptide may contain many types of modifications. Modifications can occur anywhere in a protein or polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins—Structure And Molecular Properties, 2nd Ed., T. E. Creighton, W. H. Freeman and Company, New York (1993) and Wold, F., “Posttranslational Protein Modifications: Perspectives and Prospects,” pgs. 1-12 in Posttranslational Covalent Modification Of Proteins, B. C. Johnson, Ed., Academic Press, New York (1983); Seifter et al., Meth. Enzymol. 182:626-646 (1990) and Rattan et al., “Protein Synthesis: Posttranslational Modifications and Aging,” Ann. N.Y. Acad. Sci. 663: 48-62 (1992). The term “derivatives” include chemical modifications resulting in the protein or polypeptide becoming branched or cyclic, with or without branching. Cyclic, branched and branched circular proteins or polypeptides may result from post-translational natural processes and may be made by entirely synthetic methods, as well.

The term “homologue” refers to a protein that is at least 60 percent identical in its amino acid sequence of FT as determined by standard methods that are commonly used to compare the similarity in position of the amino acids of two polypeptides. The degree of similarity or identity between two proteins can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M Stockton Press, New York, 1991; and Carillo H. and Lipman, D., SIAM, J. Applied Math., 48:1073 (1988). Preferred methods to determine identity are designed to give the largest match between the sequences tested. Methods to determine identity and similarity are codified in publicly available computer programs.

Preferred computer program methods useful in determining the identity and similarity between two sequences include, but are not limited to, the GCG program package (Devereux, J., et al., Nucleic Acids Research, 12(1): 387 (1984)), BLASTP, BLASTN, and FASTA, Atschul, S. F. et al., J. Molec. Biol., 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol., 215: 403-410 (1990). By way of example, using a computer algorithm such as GAP (Genetic Computer Group, University of Wisconsin, Madison, Wis.), the two proteins or polypeptides for which the percent sequence identity is to be determined are aligned for optimal matching of their respective amino acids (the “matched span”, as determined by the algorithm).

A gap opening penalty (which is calculated as 3 times the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al. in: Atlas of Protein Sequence and Structure, vol. 5, supp.3 for the PAM250 comparison matrix; see Henikoff et al., Proc. Natl. Acad. Sci USA, 89:10915-10919 for the BLOSUM 62 comparison matrix) also may be used by the algorithm. The percent identity then is calculated by the algorithm. Homologues will typically have one or more amino acid substitutions, deletions, and/or insertions as compared with the comparison protein or peptide, as the case may be.

The term “fusion protein” refers to a protein where one or more peptides are recombinantly fused or chemically conjugated (including covalently and non-covalently) to a protein such as (but not limited to) an antibody or antibody fragment like an Fab fragment or short chain Fv. The term “fusion protein” also refers to multimers (i.e. dimers, trimers, tetramers and higher multimers) of peptides. Such multimers comprise homomeric multimers comprising one peptide, heteromeric multimers comprising more than one peptide, and heteromeric multimers comprising at least one peptide and at least one other protein. Such multimers may be the result of hydrophobic, hyrdrophilic, ionic and/or covalent associations, bonds or links, may be formed by cross-links using linker molecules or may be linked indirectly by, for example, liposome formation.

The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. Examples of peptide mimetics in this broader sense (where part of a peptide is replaced by a structure lacking peptide bonds) are described below. Whether completely or partially non-peptide, peptide mimetics according to the embodiments provide a spatial arrangement of reactive chemical moieties that closely resemble the three-dimensional arrangement of active groups in the peptide on which the peptide mimetic is based. As a result of this similar active-site geometry, the peptide mimetic has effects on biological systems that are similar to the biological activity of the peptide.

The peptide mimetics of the embodiments are preferably substantially similar in both three-dimensional shape and biological activity to the peptides described herein. Examples of methods of structurally modifying a peptide known in the art to create a peptide mimetic include the inversion of backbone chiral centers leading to D-amino acid residue structures that may, particularly at the N-terminus, lead to enhanced stability for proteolytical degradation without adversely affecting activity. An example is given in the paper “Tritriated D-ala.sup.1-Peptide T Binding”, Smith C. S. et al., Drug Development Res., 15, pp. 371-379 (1988). A second method is altering cyclic structure for stability, such as N to C interchain imides and lactames (Ede et al. in Smith and Rivier (Eds.) “Peptides: Chemistry and Biology”, Escom, Leiden (1991), pp. 268-270). An example of this is given in conformationally restricted thymopentin-like compounds, such as those disclosed in U.S. Pat. No. 4,457,489 (1985), Goldstein, G. et al., the disclosure of which is incorporated by reference herein in its entirety. A third method is to substitute peptide bonds in the peptide by pseudopeptide bonds that. confer resistance to proteolysis.

A number of pseudopeptide bonds have been described that in general do not affect peptide structure and biological activity. One example of this approach is to substitute retro-inverso pseudopeptide bonds (“Biologically active retroinverso analogues of thymopentin”, Sisto A. et al in Rivier, J. E. and Marshall, G. R. (eds) “Peptides, Chemistry, Structure and Biology”, Escom, Leiden (1990), pp. 722-773) and Dalpozzo, et al. (1993), Int. J. Peptide Protein Res., 41:561-566, incorporated herein by reference). According to this modification, the amino acid sequences of the peptides may be identical to the sequences of an peptide described above, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus. Further modifications also can be made by replacing chemical groups of the amino acids with other chemical groups of similar structure. Another suitable pseudopeptide bond that is known to enhance stability to enzymatic cleavage with no or little loss of biological activity is the reduced isostere pseudopeptide bond (Couder, et al. (1993), Int. J. Peptide Protein Res., 41:181-184, incorporated herein by reference in its entirety).

Thus, the amino acid sequences of these peptides may be otherwise identical to the sequence of FT, except that one or more of the peptide bonds are replaced by an isostere pseudopeptide bond. Preferably the most N-terminal peptide bond is substituted, since such a substitution would confer resistance to proteolysis by exopeptidases acting on the N-terminus. The synthesis of peptides with one or more reduced isostere pseudopeptide bonds is known in the art (Couder, et al. (1993), cited above). Other examples include the introduction of ketomethylene or methylsulfide bonds to replace peptide bonds.

Peptoid derivatives of peptides represent another class of peptide mimetics that retain the important structural determinants for biological activity, yet eliminate the peptide bonds, thereby conferring resistance to proteolysis (Simon, et al., 1992, Proc. Natl. Acad. Sci. USA, 89:9367-9371, incorporated herein by reference in its entirety). Peptoids are oligomers of N-substituted glycines. A number of N-alkyl groups have been described, each corresponding to the side chain of a natural amino acid (Simon, et al. (1992), cited above). Some or all of the amino acids of the peptides may be replaced with the N-substituted glycine corresponding to the replaced amino acid.

The term “peptide mimetic” or “mimetic” also includes reverse-D peptides and enantiomers as defined below.

The term “reverse-D peptide” refers to a biologically active protein or peptide consisting of D-amino acids arranged in a reverse order as compared to the L-amino acid sequence of an peptide. Thus, the carboxy terminal residue of an L-amino acid peptide becomes the amino terminal for the D-amino acid peptide and so forth. For example, the peptide, ETESH, becomes H_{d}S_{d}E_{d}T_{d}E_{d}, where E_{d}, H_{d}, S_{d}, and T_{d} are the D-amino acids corresponding to the L-amino acids, E, H, S, and T respectively.

The term “enantiomer” refers to a biologically active protein or peptide where one or more the L-amino acid residues in the amino acid sequence of an peptide is replaced with the corresponding D-amino acid residue(s).

A “composition” as used herein, refers broadly to any composition containing FT and, optionally an additional active agent. The composition may comprise a dry formulation, an aqueous solution, or a sterile composition. Compositions comprising FT may be employed as hybridization probes. The probes may be stored in freeze-dried form and may be associated with a stabilizing agent such as a carbohydrate. In hybridizations, the probe may be deployed in an aqueous solution containing salts, e.g., NaCl, detergents, e.g., sodium dodecyl sulfate (SDS), and other components, e.g., Denhardt's solution, dry milk, salmon sperm DNA, etc.

In an embodiment in which an additional active agent is used together with FT, the expression “active agent” is used to denote any agent capable of removing unwanted cellular proliferations and/or tissue growth. Suitable active agents may include, but are not limited to: (i) anti-cancer active agents (such as alkylating agents, topoisomerase I inhibitors, topoisomerase II inhibitors, RNA/DNA antimetabolites, and antimitotic agents); (ii) active agents for treating benign growths such as anti-acne and anti-wart active agents; (iii) antiandrogen compounds, (cyproterone acetate (1a, 2ß-methylene-6-chloro-17α-acetoxy-6-dehydroprogesterone) Tamoxifen, aromatase inhibitors); (iv) alpha1-adrenergic receptor blockers (tamsulosin, terazosin, doxazosin, prazosin, bunazosin, indoramin, alfulzosin, silodosin); (v) 5α-reductase inhibitors (finasteride, dutasteride); (vi) phosphodiesterase type 5 (PDE5) inhibitors (tadalafil) and combinations thereof.

This disclosure also is premised in part on the discovery that the use of FT either alone or in combination with an additional active agent capable of treating and/or killing unwanted cellular proliferations in mammals, provides an unexpected improvement in patients suffering from obstructive voiding symptoms associated with LUTS. While not intending on being bound by any particular theory or operation, the inventor unexpectedly discovered that administration of FT to a mammal provided an unexpectedly superior improvement in patients having LUTS. The inventor unexpectedly discovered when conducting clinical trials for treating BPH, that the administration of FT, alone or in combination with another active agent, dramatically improved symptoms in patients with LUTS, including both irritative and obstructive voiding symptoms.

Patients treated with the compositions described herein exhibited a dramatic improvement in the mean obstructive voiding symptoms (MOVS) measured by the International Prostate Symptom Score (IPSS), by more than 30%, when compared to administering a control composition that does not contain FT. The embodiments may result in an improvement in the mean obstructive voiding symptoms (MOVS) measured by IPSS, within the range of from about 30% to about 150%, or from about 35% to about 125%, or from about 45% to about 105%. Patients treated with the compositions described herein exhibited a dramatic improvement in the mean irritative storage symptoms (MISS) measured IPSS, by more than 20%, when compared to administering a control composition that does not contain FT. The embodiments may result in an improvement in the mean irritative storage symptoms (MISS) measured by IPSS, within the range of from about 20% to about 50%, or from about 25% to about 40%, or from about 30% to about 35%.

The inventor discovered that the administration of conventional BPH oral medications to patients having LUTS exhibited a dramatic decrease in mean obstructive voiding symptoms (MOVS) measured by IPSS, and in mean irritative storage symptoms (MISS) measured IPSS, when compared to patients who received only placebo. The inventors discovered that the decrease in MOVS was about 50% and the decrease in MISS was about 40%. Accordingly, conventional therapies for treating BPH would not have been expected to improve symptoms of patients having LUTS, thereby making the dramatic improvement realized by the present invention that includes administration of FT even more unexpected.

The embodiments include a method of treating a mammal having LUTS, comprising administering once or more than once FT to the mammal, either alone or in combination with administration of an additional active agent. The method includes, but is not limited to, administering composition comprising FT intramuscularly, orally, intravenously, intraperitoneally, intracerebrally (intraparenchymally), intracerebroyentricularly, intralesionally, intraocularly, intraarterially, intrathecally, intratumorally, intranasally, topically, transdermally, subcutaneously, or intradermally, either alone or conjugated to a carrier.

Any mammal can benefit from use of the invention, including humans, mice, rabbits, dogs, sheep and other livestock, any mammal treated or treatable by a veterinarian, zoo-keeper, or wildlife preserve employee. Preferred mammals are humans, sheep, and dogs. Throughout this description mammals and patients are used interchangeably.

It will be apparent to one of skill in the art that other smaller fragments of FT may be selected such that these peptides will possess the same or similar biological activity. Other fragments of may be selected by one skilled in the art such that these peptides will possess the same or similar biological activity. The peptides of the embodiments encompass these other fragments. In general, the peptides of the embodiments have at least 4 amino acids, preferably at least 5 amino acids, and more preferably at least 6 amino acids.

The embodiments also encompass methods of treating mammals (or patients) having LUTS comprising administering a composition comprising FT that includes two or more FT sequences joined together, together with an additional active agent. To the extent that FT has the desired biological activity, it follows that two or more FT sequences would also possess the desired biological activity.

FT and fragments, variants, derivatives, homologues, fusion proteins and mimetics thereof encompassed by this embodiment can be prepared using methods known to those of skill in the art, such as recombinant DNA technology, protein synthesis and isolation of naturally occurring peptides, proteins, variants, derivatives and homologues thereof. FT and fragments, variants, derivatives, homologues, fusion proteins and mimetics thereof can be prepared from other peptides, proteins, and fragments, variants, derivatives and homologues thereof using methods known to those having skill in the art. Such methods include (but are not limited to) the use of proteases to cleave the peptide, or protein into FT. Any method disclosed in, for example, U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031, can be used to prepare the FT peptide described herein.

The present embodiments are directed to methods of treating mammals with LUTS, which the treatment improves the obstructive and/or irritative symptoms associated with LUTS, as measured by IPSS. Such a method comprises administering to a mammal in need thereof, a therapeutically effective amount of FT, either alone, or in combination with an additional active agent. The mammals in need may be mammals having LUTS, irrespective of mammals also having benign prostatic hyperplasia (BPH), or the mammals in need are mammals having both BPH and LUTS. The mammals in need also may be any mammal that would benefit from an improvement in symptoms associated with LUTS.

The additional active agent, if used, can be one or more active agents selected from (i) anti-cancer active agents (such as alkylating agents, topoisomerase I inhibitors, topoisomerase II inhibitors, RNA/DNA antimetabolites, and antimitotic agents); (ii) active agents for treating benign growths such as anti-acne and anti-wart active agents (salicylic acid); (iii) antiandrogen compounds, (cyproterone acetate (1α, 2ß-methylene-6-chloro-17α-acetoxy-6-dehydroprogesterone)) Tamoxifen, aromatase inhibitors); (iv) alpha1-adrenergic receptor blockers (tamsulosin, terazosin, doxazosin, prazosin, bunazosin, indoramin, alfulzosin, silodosin); (v) 5α-reductase inhibitors (finasteride, dutasteride); (vi) phosphodiesterase type 5 (PDE5) inhibitors (tadalafil) and combinations thereof. Preferably, the additional active agent is selected from the group consisting of tamsulosin, finasteride, terazosin, doxazosin, prazosin, tadalafil, alfuzosin, silodosin, dutasteride, combinations of dutasteride and tamsulosin, and mixtures and combinations thereof.

Therapeutic compositions described herein may comprise a therapeutically effective amount of FT in admixture with a pharmaceutically acceptable carrier. In some alternative embodiments, the additional active agent can be administered in the same composition with FT, and in other embodiments, the composition comprising FT is administered as an injection, whereas the additional active agent is formulated into an oral medication (gel, capsule, tablet, liquid, etc.). The carrier material may be water for injection, preferably supplemented with other materials common in solutions for administration to mammals. Typically, FT will be administered in the form of a composition comprising the purified FT peptide in conjunction with one or more physiologically acceptable carriers, excipients, or diluents. Neutral buffered saline or saline mixed with serum albumin are exemplary appropriate carriers. Preferably, the product is formulated as a lyophilizate using appropriate excipients (e.g., sucrose). Other standard carriers, diluents, and excipients may be included as desired. Compositions of the embodiments also may comprise buffers known to those having ordinary skill in the art with an appropriate range of pH values, including Tris buffer of about pH 7.0-8.5, or acetate buffer of about pH 4.0-5.5, which may further include sorbitol or a suitable substitute therefor.

Solid dosage forms for oral administration include but are not limited to, capsules, tablets, pills, powders, and granules. In such solid dosage forms, the additional active agent, and/or FT can be admixed with at least one of the following: (a) one or more inert excipients (or carrier), such as sodium citrate or dicalcium phosphate; (b) fillers or extenders, such as starches, lactose, sucrose, glucose, mannitol, and silicic acid; (c) binders, such as carboxymethylcellulose, alginates, gelatin, polyvinylpyrrolidone, sucrose and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as acetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the active compounds, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Actual dosage levels of active ingredients in the compositions of the embodiments may be varied to obtain an amount of FT and additional active agent that is effective to obtain a desired therapeutic response for a particular composition and method of administration. The selected dosage level therefore depends upon the desired therapeutic effect, the route of administration, the desired duration of treatment, and other factors.

With mammals, including humans, the effective amounts can be administered on the basis of body surface area. The interrelationship of dosages for animals of various sizes, species and humans (based on mg/M² of body surface) is described by E. J. Freireich et al., Cancer Chemother. Rep., 50 (4):219 (1966). Body surface area may be approximately determined from the height and weight of an individual (see e.g., Scientific Tables, Geigy Pharmaceuticals, Ardsley, N.Y. pp. 537-538 (1970)).

The total daily dose of the FT peptide and additional active agent administered to a host may be in single or divided doses. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, potency of the administered drug, rates of absorption and excretion, combination with other drugs and the severity of the particular disease being treated. It is preferred that the composition is administered only once as an injection or infusion, or in another preferred embodiment, the composition is administered twice. In this embodiment, the period of time between administration of the composition may vary anywhere from 2 months to 10 years, or from 8 months to 4 years, or more than about one year (e.g., between 1 and 2 years).

A method of administering a composition comprising FT according to the embodiments includes, but is not limited to, administering the compositions intramuscularly, orally, intravenously, intraperitoneally, intracerebrally (intraparenchymally), intracerebroventricularly, intratumorally, intralesionally, intradermally, intrathecally, intranasally, intraocularly, intraarterially, topically, transdermally, via an aerosol, infusion, bolus injection, implantation device, sustained release system etc. Any method of administration disclosed in, for example, U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031, can be used.

In certain embodiments, the invention provides a method of improving symptoms in mammals having LUTS, optionally including mammals also having BPH, which comprises identifying a mammal having BPH who optionally also has LUTS, and administering to the mammal at least once, a therapeutically effective amount of FT, (SEQ ID NO. 1 (Ile-Asp-Gln-Gln-Val-Leu-Ser-Arg-Ile-Lys-Leu-Glu-Ile-Lys-Arg-Cys-Leu)). In certain embodiments, the isolated FT peptide can be administered in combination with at least one active agent selected from the group consisting of (1) of an inhibitor of 5α-reductase and/or an antiestrogen, (2) an inhibitor of 5α-reductase and/or an aromatase inhibitor, (3) a 5α-reductase inhibitor and/or a 17β-HSD inhibitor, (4) a 5α-reductase inhibitor, an antiestrogen and an aromatase inhibitor, (5) a 5α-reductase inhibitor, an antiestrogen and a 17β-HSD inhibitor, (6) a 5α-reductase inhibitor, an aromatase inhibitor, an antiestrogen and a 17β-HSD inhibitor, (7) a 5α-reductase inhibitor, an antiandrogen and an antiestrogen, (8), a 5α-reductase inhibitor, an antiandrogen and an aromatase inhibitor, (9) a 5α-reductase inhibitor, an antiandrogen and an 17β-HSD inhibitor, (10) a 5α-reductase inhibitor, an antiandrogen, an antiestrogen and an aromatase inhibitor, (11) a 5α-reductase inhibitor, an antiandrogen, an aromatase inhibitor and a 17β-HSD inhibitor, (12) a 5α-reductase inhibitor, an antiandrogen, an aromatase inhibitor, an antiestrogen and a 17β-HSD inhibitor, (13) a 17β-HSD inhibitor and an antiestrogen, (14) a 17β-HSD inhibitor and an aromatase inhibitor, (15) a 17β-HSD inhibitor, an aromatase inhibitor and an antiestrogen, (16) a 17β-HSD inhibitor, an antiandrogen and an antiestrogen, (17) a 17β-HSD inhibitor, an antiandrogen and an aromatase inhibitor, (18) a 17β-HSD inhibitor, an antiandrogen, an antiestrogen and an aromatase inhibitor, (19) an antiestrogen and an aromatase inhibitor and (20) an antiestrogen, an aromatase inhibitor, and an antiandrogen, (21) an LHRH agonist or antagonist, an inhibitor of 5α-reductase and an antiestrogen, (22) an LHRH agonist or antagonist, an inhibitor of 5α-reductase and an aromatase inhibitor, (23) an LHRH agonist or antagonist, a 5a reductase inhibitor and a 17β-HSD inhibitor, (24) an LHRH agonist or antagonist, a 5α-reductase inhibitor, an antiestrogen and an aromatase inhibitor, (25) an LHRH agonist or antagonist, a 5α-reductase inhibitor, an antiestrogen and a 17β-HSD inhibitor, (26) an LHRH agonist or antagonist, a 5α-reductase inhibitor, an aromatase inhibitor, an antiestrogen and a 17β-HSD inhibitor, (27) an LHRH agonist or antagonist, a 5α-reductase inhibitor, an antiandrogen and an antiestrogen, (28), an LHRH agonist or antagonist, a 5α-reductase inhibitor, an antiandrogen and an aromatase inhibitor, (29) an LHRH agonist or antagonist, a 5α-reductase inhibitor, an antiandrogen and an 17β-HSD inhibitor, (30) an LHRH agonist or antagonist, a 5α-reductase inhibitor, an antiandrogen, an antiestrogen and an aromatase inhibitor, (31) an LHRH agonist or antagonist, a 5α-reductase inhibitor, an antiandrogen, an aromatase inhibitor and a 17β-HSD inhibitor, (32) an LHRH agonist or antagonist, a 5α-reductase inhibitor, an antiandrogen, an aromatase inhibitor, an antiestrogen and a 17β-HSD inhibitor, (33) an LHRH agonist or antagonist, a 17β-HSD inhibitor and an antiestrogen, (34) an LHRH agonist or antagonist, a 17β-HSD inhibitor and an aromatase inhibitor, (35) an LHRH agonist or antagonist, a 17β-HSD inhibitor, an aromatase inhibitor and an antiestrogen, (36) an LHRH agonist or antagonist, a 17β-HSD inhibitor, an antiandrogen and an antiestrogen, (37) an LHRH agonist or antagonist, a 17β-HSD inhibitor, an antiandrogen and an aromatase inhibitor, (38) an LHRH agonist or antagonist, a 17β-HSD inhibitor, an antiandrogen, an antiestrogen and an aromatase inhibitor, (39) an LHRH agonist or antagonist, an antiestrogen and an aromatase inhibitor and (40) an LHRH agonist or antagonist, an antiestrogen, an aromatase inhibitor, and an antiandrogen.

The following examples are provided to illustrate the present embodiments. It should be understood, however, that the embodiments are not to be limited to the specific conditions or details described in these examples. Throughout the specification, any and all references to a publicly available document, including a U.S. patent, are specifically incorporated by reference. In particular, the embodiments expressly incorporate by reference the examples contained in U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031, each of which reveal that certain peptides specified therein are effective agents for causing cell death in vivo in normal rodent muscle tissue, subcutaneous connective tissue, dermis and other tissue.

### Example One

Patients with BPH and who optionally also had LUTS were given an intraprostatic injection of either a) FT (SEQ ID NO. 1 (Ile-Asp-Gln-Gln-Val-Leu-Ser-Arg-Ile-Lys-Leu-Glu-Ile-Lys-Arg-Cys-Leu), 2.5 mg) in phosphate buffered saline pH 7.2 (“PBS”) or b) PBS alone, under double-blind conditions by a urologist in an office setting under ultrasound guidance. Patients were followed for 1 to 6 years with regular physical examinations, laboratory tests, and evaluations of symptoms. Symptomatic evaluation was measured by the International Prostate Symptom Score (IPSS) which is a quantitative scale used to gauge prostatic symptomatic improvement or worsening. The IPSS quantifies the following: 1) incomplete bladder emptying after urination; 2) frequent urination; 3) stopping and starting during urination; 4) urgent need to urinate; 5) weakness of urinary stream; 6) need to push or strain during urination; 7) need to urinate after going to sleep at night (nocturia).

IPSS items 1, 3, 5 and 6 comprise the obstructive voiding scales and IPSS items 2, 4, and 7 comprise the irritative storage scales. The difference from baseline irritative storage scores to follow-up scores; and from baseline obstructive voiding scores to follow-up scores were calculated in FT treated subjects and Placebo treated controls. Surprisingly, the amount of improvement in the obstructive voiding results in subjects who had a single prior injection of FT was significantly better than the changes found in irritative storage symptoms. The results are summarized in Table One.

TABLE 1

| | Number of | | |
|---|---|---|---|
| Treatment | patients | MISS (SD) | MOVS (SD) |
| SEQ ID NO: 1-FT | 287 | 1.89 (3.10) | 3.44 (4.90)* |
| Control | 217 | 1.41 (3.36) | 2.35 (4.71) |

*p = .012 t test vs Placebo Control.

As shown in Table 1, patients treated with the compositions described herein exhibited an improvement in the mean obstructive voiding symptoms (MOVS) measured by the International Prostate Symptom Score (IPSS), by more than 46%, when compared to administering a control composition that does not contain FT. Table 1 also shows that patients treated with the compositions described herein exhibited an improvement in the mean irritative storage symptoms (MISS) measured IPSS, by more than 34%, when compared to administering a control composition that does not contain FT. Administration of FT therefore provides a greater improvement in MOVS and MISS in patients suffering from BPH. Because patients with BPH may or may not also have LUTS, the improvement likely would be even greater in only patients suffering from both BPH and LUTS.

### Example Two

In a second analysis group, patients with BPH who also optionally had LUTS were given an intraprostatic injection of PBS pH 7.2 vehicle alone, under double-blind conditions by a urologist in an office setting under ultrasound guidance. Patients were followed for 1 to 3 years with regular physical examinations, laboratory tests, and evaluations of symptoms. Patients who received PBS vehicle alone injections and who subsequently received in addition conventional oral medications used to treat BPH including alpha blockers such as tamsulosin, terazosin, doxazosin, or 5-alpha reductase inhibitors such as finasteride, dutasteride, or phosphodiesterase type 5 inhibitors (PDE5 inhibitors) such as tadalafil, were assessed before and after receiving the placebo, and after receiving additional conventional oral BPH medications. The IPSS quantifies the following: 1) incomplete bladder emptying after urination; 2) frequent urination; 3) stopping and starting during urination; 4) urgent need to urinate; 5) weakness of urinary stream; 6) need to push or strain during urination; 7) need to urinate after going to sleep at night (nocturia). IPSS items 1, 3, 5 and 6 comprise the obstructive voiding scales and IPSS items 2, 4, and 7 comprise the irritative storage scales. The difference from baseline irritative storage scores to follow-up scores; and from baseline obstructive voiding scores to follow-up scores were calculated in blinded placebo treated patients and in blinded placebo treated patients subsequently treated with conventional oral BPH medications. The amount of improvement in the obstructive voiding and irritative storage results in subjects who received subsequent BPH oral medications after prior injection of placebo was not significantly better than, and indeed worse than the changes found in the subjects after the initial placebo injection. The results are summarized in Table Two.

TABLE 2

| | Number of | | |
|---|---|---|---|
| Treatment | patients | MISS (SD) | MOVS (SD) |
| Placebo + | 42 | 0.83 (2.66) | 1.14 (4.04) |
| conventional | | | |
| medication | | | |
| Control | 217 | 1.41 (3.36) | 2.35 (4.71) |

The results of Table 2 show that patients with BPH that receive conventional oral medication for treating BPH show no improvement in either MOVS or MISS, when compared to the patients who only receive the placebo control. Indeed, the symptoms of LUTS appear to worsen somewhat in patients who receive conventional oral medication for treating BPH. Accordingly, agents known to be effective in treating BPH would not have been expected to provide an improvement in symptoms in patients also having LUTS, much less the dramatic improvement shown herein for patients treated with FT.

### Example Three

In a third analysis group, patients with BPH who also optionally had LUTS were given an intraprostatic injection of PBS pH 7.2 vehicle alone, under double-blind conditions by a urologist in an office setting under ultrasound guidance. Patients were followed for 1 to 3 years with regular physical examinations, laboratory tests, and evaluations of symptoms. 131 patients who received PBS vehicle alone injections after 1-3 years received a cross-over injection of FT (SEQ ID NO. 1 (Ile-Asp-Gln-Gln-Val-Leu-Ser-Arg-Ile-Lys-Leu-Glu-Ile-Lys-Arg-Cys-Leu), 2.5 mg) in phosphate buffered saline pH 7.2 (“PBS”). The differences from baseline obstructive voiding scores to follow-up scores were calculated in blinded placebo treated patients (Group One); blinded placebo treated patients who received subsequent conventional oral BPH medications (Group Two), and blinded placebo treated patients who subsequently received FT treatment (Group Three). The amount of improvement in the obstructive voiding results in subjects who received subsequent FT injection after prior injection of placebo was significantly better than the changes found in the subjects after the initial placebo injection, and significantly better than the improvement in patients who received blinded placebo followed by conventional oral BPH medications. The results are summarized in Table Three below.

TABLE 3

| Treatment | Number of patients | MOVS (SD) |
|---|---|---|
| Control | 217 | 2.35 (4.71) |
| Placebo + conventional | 42 | 1.14 (4.04) |
| medication | | |
| SEQ ID NO: 1-FT | 131 | 4.80 (4.74)* ** |

*p < .0001, t test compared to placebo alone;
**p < .0001, t test compared to placebo followed by subsequent conventional oral BPH medications

As shown in Table 3, patients treated with the compositions described herein exhibited an improvement in the mean obstructive voiding symptoms (MOVS) measured by the International Prostate Symptom Score (IPSS), by more than 104%, when compared to administering a control composition that does not contain FT, and by more than 320%, when compared to administering a placebo control followed by conventional oral BPH medication. Because FT has previously been reported to be effective in treating BPH, and because known BPH therapies (e.g., conventional oral medication—see Example 2 above) were found not effective in treating either irritative or obstructive symptoms, there would have been no reason to expect that administration of FT would improve the obstructive voiding symptoms in patients with LUTS, much less a greater than 300% improvement, when compared to other known BPH therapies.

### Example Four

In a fourth analysis group, patients with BPH who also optionally had LUTS were given an intraprostatic injection of PBS pH 7.2 vehicle alone or of FT, under double-blind conditions by a urologist in an office setting under ultrasound guidance. Patients were followed for 1 to 3 years with regular physical examinations, laboratory tests, and evaluations of symptoms. 217 patients received double blind placebo alone (Group One). 287 patients received double blind FT alone (Group Two). After 1-3 years, 131 patients who received PBS vehicle alone injections received a cross-over injection of FT (Group Three). The final group of 189 patients received blinded FT followed after 1-3 years by a second injection of FT (Group Four). The difference from baseline obstructive voiding scores to follow-up scores were calculated in blinded placebo treated patients (n=217), and compared to groups 2-4 who received FT treatment (n=607). The amount of improvement in the obstructive voiding results in all subjects who received FT injection was significantly better than the changes found in the subjects after placebo injection alone. The results are summarized in Table Four.

TABLE 4

| Treatment | Number of patients | MOVS (SD) |
|---|---|---|
| SEQ ID NO: 1-FT | 607 | 4.82 (4.94)* |
| Control | 217 | 2.35 (4.71) |

*p < 0.0001, t test vs Placebo Control.

As shown in Table 4, patients treated with the compositions described herein exhibited an improvement in the mean obstructive voiding symptoms (MOVS) measured by the International Prostate Symptom Score (IPSS), by more than 105%, when compared to administering a control composition that does not contain FT.

The results from the foregoing examples illustrate the unexpectedly superior effect of the FT peptide, in improving the symptoms in patients with LUTS. It will be apparent to those skilled in the art that various modifications and variations can be made in the methods and compositions of the present embodiments without departing from the spirit or scope of the embodiments.

1. A method of improving the symptoms of mammals having Lower Urinary Tract Symptoms (LUTS) comprising: (i) identifying mammals having LUTS and do not have benign prostatic hyperplasia (BPH); and (ii) administering to the mammal having LUTS but not BPH a therapeutically effective amount of SEQ ID NO. 1 (Ile-Asp-Gln-Gln-Val-Leu-Ser-Arg-Ile-Lys-Leu-Glu-Ile-Lys-Arg-Cys-Leu), wherein the method improves the mean obstructive voiding symptoms (MOVS) measured by the International Prostate Symptom Score (IPSS), by more than 30%, when compared to administering a control composition that does not contain SEQ ID NO. 1.

2. The method of claim 1, wherein the method comprises administering a therapeutically effective amount of SEQ ID NO. 1 as claimed in claim 1 and a carrier.

3. The method of claim 1, wherein SEQ ID NO. 1 is administered more than once.

4. The method of claim 1, wherein SEQ ID NO. 1 is administered by a method selected from the group consisting of orally, subcutaneously, intradermally, intranasally, intravenously, intramuscularly, intrathecally, intranasally, intratumorally, topically, and transdermally.

5. The method of claim 1, wherein the method improves the mean obstructive voiding symptoms (MOVS) measured by IPSS, within the range of from about 30% to about 150%.

6. The method of claim 5, wherein the method improves the mean obstructive voiding symptoms (MOVS) measured by IPSS, within the range of from about 35% to about 125%.

7. The method of claim 6, wherein the method improves the mean obstructive voiding symptoms (MOVS) measured by IPSS, within the range of from about 45% to about 105%.

8. The method of claim 1, wherein the method improves the mean obstructive voiding symptoms (MOVS) measured by IPSS, by more than 300%, when compared to the MOVS of patients who received oral medication known to be useful in treating benign prostatic hyperplasia (BPH).

9. The method of claim 1, wherein the method improves the mean irritative storage symptoms (MISS) measured by IPSS, by more than 20%, when compared to administering a control composition that does not contain SEQ ID NO. 1.

10. The method of claim 9, wherein the method improves the mean irritative storage symptoms (MISS) measured by IPSS by an amount within the range of from about 20% to about 50%.

11. The method of claim 10, wherein the method improves the mean irritative storage symptoms (MISS) measured by IPSS by an amount within the range of from about 25% to about 40%.

12. The method of claim 11, wherein the method improves the mean irritative storage symptoms (MISS) measured by IPSS by an amount within the range of from about 30% to about 35%.
